(19) United States
(12) Patent Application Publication
(10) Pub. No.: US 2026/0053786 A1
(43) Pub. Date: Feb. 26, 2026

(54) FORMULATIONS OF RADIPRODIL

(71) Applicant: GRIN Therapeutics, Inc., New York, NY (US)

(72) Inventors: Marie Genin, New York, NY (US); Pierandrea Muglia, Brussels (BE)

(21) Appl. No.: 19/376,486

(22) Filed: Oct. 31, 2025

Related U.S. Application Data
(63) Continuation of application No. 18/681,382.
(60) Provisional application No. 63/230,331, filed on Aug. 6, 2021.

(51) Int. Cl. A61K 31/454; A61K 9/16

(52) U.S. Cl. CPC A61K 31/454; A61K 9/1623; A61K 9/1652

(57) ABSTRACT

The present disclosure provides, in part, pharmaceutical compositions comprising radiprodil and pharmaceutically acceptable excipients and methods of use thereof in the treatment of disorders such as epileptic disorders.

### CROSS-REFERENCE

This application claims priority to U.S. Provisional Application No. 63/230,331 filed Aug. 6, 2021, which is incorporated herein by reference in its entirety.

### BACKGROUND

N-methyl-D-aspartate (NMDA) receptors are ligand-gated cation-channels embedded in the cell membranes of neurons. Overactivation of NMDA receptors by glutamate, their natural ligand, can lead to calcium overload of cells. This triggers a cascade of intracellular events that alters the cell function and ultimately may lead to death of neurons. Modulators of the NMDA receptors may be used for treating many disorders that are accompanied with excess release of glutamate, the main excitatory neurotransmitter in the central nervous system. For example, NR2B subtype selective antagonists of NMDA receptors are expected to possess little or no untoward side effects that are typically caused by the non-selective antagonists of NMDA receptors, namely psychotomimetic effects such as dizziness, headache, hallucinations, dysphoria and disturbances of cognitive and motor function. There is a need for NMDA receptor modulators that are useful for the treatment of disorders.

### SUMMARY

The present disclosure provides, in an embodiment, compositions comprising radiprodil, and methods of use thereof.

In one embodiment, described herein is a pharmaceutically acceptable composition formulated for oral administration of a compound of Formula I:

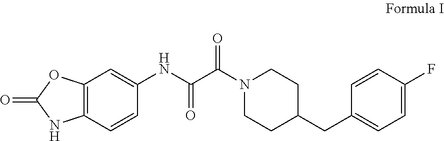 [SMILES: O=C(Nc1ccc2[nH]c(=O)oc2c1)C(=O)N1CCC(Cc2ccc(F)cc2)CC1]

comprising: about 0.5% by weight to about 15% by weight of the compound of Formula I based on the total weight of the composition; at least one filler; a disintegrant; a binder; and a surfactant.

In some embodiments, compositions described herein comprise Form A of the compound of Formula I. Form A may be characterized by an X-ray powder diffraction pattern with characteristic peaks between and including the following values of 2θ in degrees: 7.8, 22.0, 23.7, 27.0 and 27.6±0.2° 2θ.

In some embodiments, compositions described herein comprise Form C of the compound of Formula I. Form C may be characterized by an X-ray powder diffraction pattern with characteristic peaks between and including the following values of 2θ in degrees: 6.4, 13.7, and 25.8±0.2° 2θ.

In one embodiment, provided herein is a solid pharmaceutically acceptable composition formulated for oral administration of a compound of Formula I:

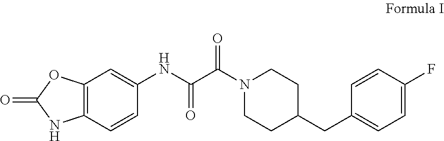 [SMILES: O=C(Nc1ccc2[nH]c(=O)oc2c1)C(=O)N1CCC(Cc2ccc(F)cc2)CC1]

comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the

In one embodiment, described herein is a solid pharmaceutically acceptable composition formulated for oral administration of a compound of Formula I:

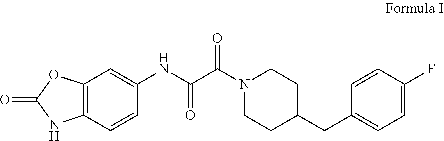 [SMILES: O=C(Nc1ccc2[nH]c(=O)oc2c1)C(=O)N1CCC(Cc2ccc(F)cc2)CC1]

comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition.

In one embodiment, described herein is a pharmaceutically acceptable composition formulated for oral administration of a compound of Formula I:

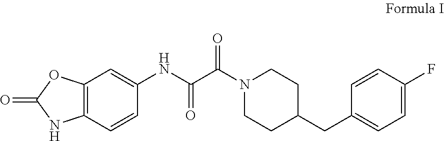 [SMILES: O=C(Nc1ccc2[nH]c(=O)oc2c1)C(=O)N1CCC(Cc2ccc(F)cc2)CC1]

comprising: about 0.5% to 15% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; not more than about 0.1% to 0.5% of an impurity (e.g., 6-amino-2-benzoxazolone) with respect to the quantity of the compound as measured by HPLC; and one or more pharmaceutically acceptable excipients.

In one embodiment, provided herein is a pharmaceutically acceptable aqueous suspension comprising a pharmaceutically acceptable composition described herein and an aqueous medium.

In one embodiment, described herein is a pharmaceutically acceptable aqueous suspension for orally delivering about 0.1 mg/kg to 2 mg/kg of a compound of Formula I:

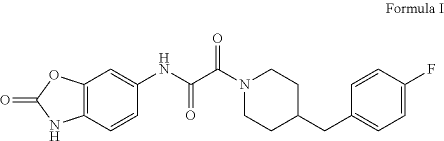 [SMILES: O=C(Nc1ccc2[nH]c(=O)oc2c1)C(=O)N1CCC(Cc2ccc(F)cc2)CC1]

comprising: (i) a solid pharmaceutically acceptable composition comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium.

In one embodiment, provided herein is a pharmaceutically acceptable aqueous suspension for orally delivering about 0.2 mg/kg to 2 mg/kg of a compound of Formula I:

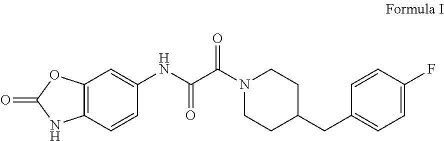 [SMILES: O=C(Nc1ccc2[nH]c(=O)oc2c1)C(=O)N1CCC(Cc2ccc(F)cc2)CC1]

(i) a solid pharmaceutically acceptable composition comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium. In some embodiments, the aqueous medium comprises a starch-based suspension (e.g., SYRSPEND® SF).

In one embodiment, provided herein is a solid pharmaceutically acceptable composition formulated for oral administration of a compound of Formula I:

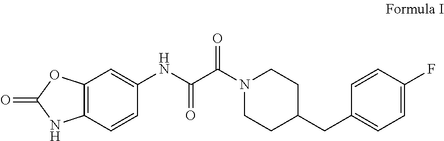 [SMILES: O=C(Nc1ccc2[nH]c(=O)oc2c1)C(=O)N1CCC(Cc2ccc(F)cc2)CC1]

comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition.

In one embodiment, described herein is a solid pharmaceutically acceptable composition formulated for oral administration of a compound of Formula I:

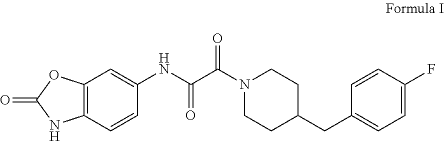 [SMILES: O=C(Nc1ccc2[nH]c(=O)oc2c1)C(=O)N1CCC(Cc2ccc(F)cc2)CC1]

comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition.

In one embodiment, described herein is a pharmaceutically acceptable composition formulated for oral administration of a compound of Formula I:

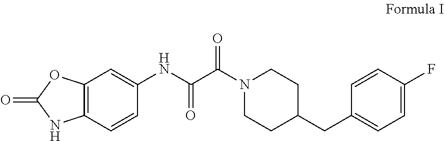 [SMILES: O=C(Nc1ccc2[nH]c(=O)oc2c1)C(=O)N1CCC(Cc2ccc(F)cc2)CC1]

comprising: about 0.5% to 15% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; not more than about 0.1% to 0.5% of an impurity (e.g., 6-amino-2-benzoxazolone) with respect to the quantity of the compound as measured by HPLC; and one or more pharmaceutically acceptable excipients.

In one embodiment, provided herein is a pharmaceutically acceptable aqueous suspension comprising a pharmaceutically acceptable composition described herein and an aqueous medium.

In one embodiment, described herein is a pharmaceutically acceptable aqueous suspension for orally delivering about 0.1 mg/kg to 2 mg/kg of a compound of Formula I:

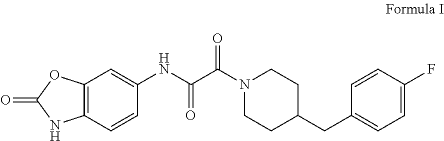 [SMILES: O=C(Nc1ccc2[nH]c(=O)oc2c1)C(=O)N1CCC(Cc2ccc(F)cc2)CC1]

comprising: (i) a solid pharmaceutically acceptable composition comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium.

In one embodiment, provided herein is a pharmaceutically acceptable aqueous suspension for orally delivering about 0.2 mg/kg to 2 mg/kg of a compound of Formula I:

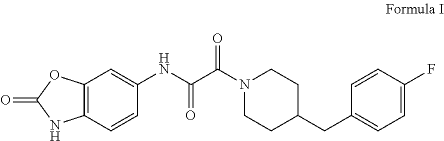 [SMILES: O=C(Nc1ccc2[nH]c(=O)oc2c1)C(=O)N1CCC(Cc2ccc(F)cc2)CC1]

(i) a solid pharmaceutically acceptable composition comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium. In some embodiments, the aqueous medium comprises a starch-based suspension (e.g., SYRSPEND® SF).

### BRIEF DESCRIPTION OF THE DRAWINGS

FIG. 1 depicts an exemplary manufacturing process of 1 weight % and 10 weight % radiprodil granules.

FIG. 2 depicts dissolution profiles for exemplary radiprodil samples that were not reconstituted prior to dissolution testing.

FIG. 3 depicts dissolution profiles for exemplary radiprodil samples that were reconstituted prior to dissolution testing.

FIG. 4 depicts dissolution profiles for exemplary radiprodil samples that underwent extemporaneous reconsistution prior to dissolution testing.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one embodiment, provided herein is a compound of the formula:

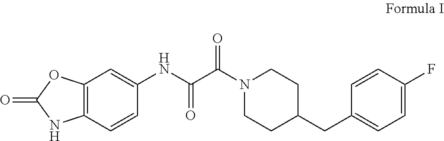 [SMILES: O=C(Nc1ccc2[nH]c(=O)oc2c1)C(=O)N1CCC(Cc2ccc(F)cc2)CC1]

or a pharmaceutically acceptable salt thereof.

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The pharmaceutically acceptable compositions described herein may comprise one or more impurities, such as those described herein. Exemplary impurities include compounds listed in Table 7 as provided herein.

In one embodiment, described herein is a pharmaceutically acceptable composition formulated for oral administration of a compound of Formula I:

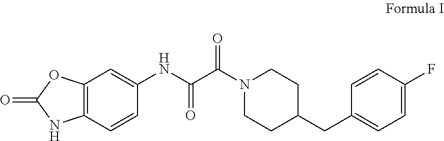 [SMILES: O=C(Nc1ccc2[nH]c(=O)oc2c1)C(=O)N1CCC(Cc2ccc(F)cc2)CC1]

comprising: about 0.5% by weight to about 15% by weight of the compound of Formula I based on the total weight of the composition; at least one filler; a disintegrant; a binder; and a surfactant.

In some embodiments, the composition comprises about 1% by weight of the compound based on the total weight of the pharmaceutically acceptable composition. In some embodiments, the composition comprises about 10% by weight of the compound based on the total weight of the pharmaceutically acceptable composition. In some embodiments, the composition comprises an anhydrous crystalline form of the compound of Formula I. In some embodiments, the anhydrous crystalline form has an X-ray powder diffraction pattern with characteristic peaks between and including the following values of 2θ in degrees: 7.8, 22.0, 23.7, 27.0 and 27.6±0.2° 2θ. In some embodiments, the pharmaceutical composition comprises about 10% by weight to about 80% by weight of at least one filler based on the total weight of the pharmaceutical composition. In some embodiments, the at least one filler is selected from the group consisting of confectioner's sugar, compressible sugar, dextrates, dextrin, dextrose, lactose, mannitol, microcrystalline cellulose, powdered cellulose, sorbitol, sucrose, talc, and combinations thereof. In some embodiments, the composition comprises two fillers. In some embodiments, the pharmaceutical composition comprises about 1% by weight to about 10% by weight of the disintegrant based on the total weight of the pharmaceutical composition. In some embodiments, the disintegrant is selected from the group consisting of crospovidone, croscarmellose sodium, sodium starch glycolate, microcrystalline cellulose, pregelatinized starch, and combinations thereof. In some embodiments, the pharmaceutical composition comprises about 1% by weight to about 10% by weight of the binder based on the total weight of the pharmaceutical composition.

In some embodiments, the binder is selected from the group consisting of povidone, starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly (vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and combinations thereof. In some embodiments, the pharmaceutical composition comprises about 0.01% by weight to about 5% by weight of the surfactant based on the total weight of the pharmaceutical composition. In some embodiments, the surfactant is selected from the group consisting of polyoxyethylene stearates, polyoxyethylene alkyl ethers, sorbitan fatty acid esters, poloxamers, polyoxyethylene castor oil derivatives, phospholipids, sodium lauryl sulphate, polysorbate (polyoxyethylene sorbitan fatty acid esters), and combinations thereof. In some embodiments, the composition is a granule for an oral solution.

In one embodiment, provided herein is a solid pharmaceutically acceptable composition formulated for oral administration of a compound of Formula I:

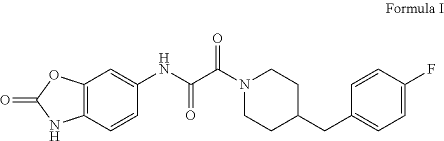 [SMILES: O=C(Nc1ccc2[nH]c(=O)oc2c1)C(=O)N1CCC(Cc2ccc(F)cc2)CC1]

comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the

In one embodiment, described herein is a solid pharmaceutically acceptable composition formulated for oral administration of a compound of Formula I:

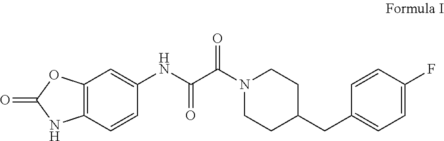 [SMILES: O=C(Nc1ccc2[nH]c(=O)oc2c1)C(=O)N1CCC(Cc2ccc(F)cc2)CC1]

comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition.

In some embodiments, the composition comprises not more than about 0.1% to 0.5% (e.g., not more than 0.05%) of an impurity with respect to the quantity of the compound as measured by HPLC. In some embodiments, the composition comprises not more than about 0.1% to 0.5% of 6-amino-2-benzoxazolone with respect to the quantity of the compound as measured by HPLC.

In one embodiment, described herein is a pharmaceutically acceptable composition formulated for oral administration of a compound of Formula I:

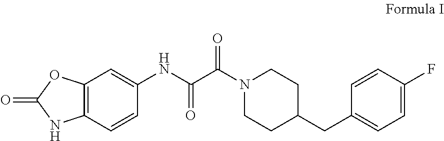 [SMILES: O=C(Nc1ccc2[nH]c(=O)oc2c1)C(=O)N1CCC(Cc2ccc(F)cc2)CC1]

comprising: about 0.5% to 15% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; not more than about 0.1% to 0.5% of an impurity (e.g., 6-amino-2-benzoxazolone) with respect to the quantity of the compound as measured by HPLC; and one or more pharmaceutically acceptable excipients.

In one embodiment, provided herein is a solid pharmaceutically acceptable composition formulated for oral administration of a compound of Formula I:

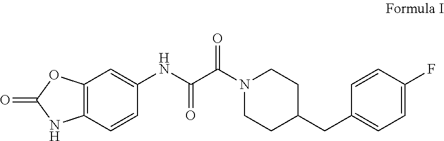 [SMILES: O=C(Nc1ccc2[nH]c(=O)oc2c1)C(=O)N1CCC(Cc2ccc(F)cc2)CC1]

comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition.

In one embodiment, described herein is a solid pharmaceutically acceptable composition formulated for oral administration of a compound of Formula I:

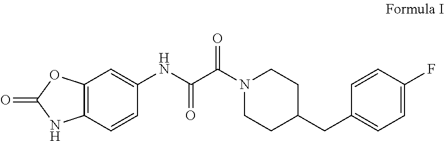 [SMILES: O=C(Nc1ccc2[nH]c(=O)oc2c1)C(=O)N1CCC(Cc2ccc(F)cc2)CC1]

comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition.

In one embodiment, described herein is a pharmaceutically acceptable composition formulated for oral administration of a compound of Formula I:

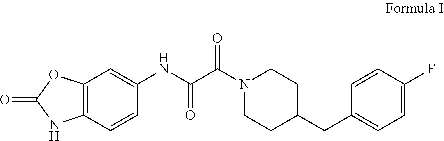 [SMILES: O=C(Nc1ccc2[nH]c(=O)oc2c1)C(=O)N1CCC(Cc2ccc(F)cc2)CC1]

comprising: about 0.5% to 15% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; not more than about 0.1% to 0.5% of an impurity (e.g., 6-amino-2-benzoxazolone) with respect to the quantity of the compound as measured by HPLC; and one or more pharmaceutically acceptable excipients. In some embodiments, the composition comprises not more than about 0.5% of an impurity (e.g., 6-amino-2-benzoxazolone) with respect to the quantity of the compound as measured by HPLC. In some embodiments, the composition comprises not more than about 0.05% of an impurity (e.g., 6-amino-2-benzoxazolone) with respect to the quantity of the compound as measured by HPLC. In some embodiments, the composition comprises not more than about 0.5% of an impurity (e.g., 6-amino-2-benzoxazolone) with respect to when exposed to 60% relative humidity at 25° C. for about 6 months. In some embodiments, the composition comprises not more than about 0.05% of an impurity (e.g., 6-amino-2-benzoxazolone) with respect to when exposed to 60% relative humidity at 25° C. for about 6 months. In some embodiments, the composition comprises not more than about 0.5% of an impurity (e.g., 6-amino-2-benzoxazolone) with respect to when exposed to 60% relative humidity at 25° C. for about 36 months. In some embodiments, the composition comprises not more than about 0.05% of an impurity (e.g., 6-amino-2-benzoxazolone) with respect to when exposed to 60% relative humidity at 25° C. for about 36 months. In some embodiments, the composition comprises about 10% of the anhydrous crystalline form based on the total weight of the composition. In some embodiments, the composition comprises about 1% of the anhydrous crystalline form based on the total weight of the composition. In some embodiments, the composition releases at least 80% of the compound after 30 minutes when the composition is tested in 900 mL sodium lauryl sulfate solution in water using a USPII Paddle Apparatus at 37° C., with a paddle speed of 75 rpm. In some embodiments, the composition releases at least 80% (e.g., at least 90% or at least 95%) of the compound when the composition is stirred in an aqueous medium from at least 1 minute to about 24 hours after reconstitution. In some embodiments, the aqueous medium comprises a starch-based suspension. In one embodiment, provided herein is a pharmaceutically acceptable aqueous suspension comprising a pharmaceutically acceptable composition described herein and an aqueous medium. In some embodiments, the aqueous medium comprising a starch-based suspension (e.g., SYRSPEND® SF).

In one embodiment, described herein is a pharmaceutically acceptable aqueous suspension for orally delivering about 0.1 mg/kg to 2 mg/kg of a compound of Formula I:

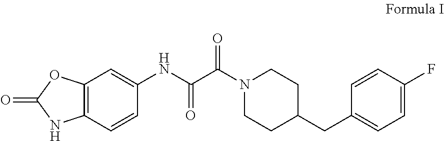 [SMILES: O=C(Nc1ccc2[nH]c(=O)oc2c1)C(=O)N1CCC(Cc2ccc(F)cc2)CC1]

comprising: (i) a solid pharmaceutically acceptable composition comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium.

In one embodiment, provided herein is a pharmaceutically acceptable aqueous suspension for orally delivering about 0.2 mg/kg to 2 mg/kg of a compound of Formula I:

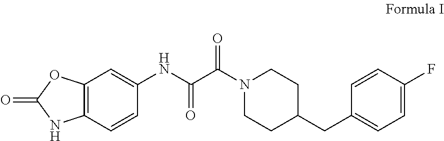 [SMILES: O=C(Nc1ccc2[nH]c(=O)oc2c1)C(=O)N1CCC(Cc2ccc(F)cc2)CC1]

(i) a solid pharmaceutically acceptable composition comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 7.8, 22.0, 23.7, 27.0 and 27.6±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium. In some embodiments, the aqueous medium comprises a starch-based suspension (e.g., SYRSPEND® SF).

In one embodiment, described herein is a pharmaceutically acceptable aqueous suspension for orally delivering about 0.1 mg/kg to 2 mg/kg of a compound of Formula I:

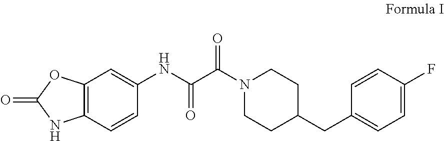 [SMILES: O=C(Nc1ccc2[nH]c(=O)oc2c1)C(=O)N1CCC(Cc2ccc(F)cc2)CC1]

comprising: (i) a solid pharmaceutically acceptable composition comprising: about 10% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 50% to 80% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium.

In one embodiment, provided herein is a pharmaceutically acceptable aqueous suspension for orally delivering about 0.2 mg/kg to 2 mg/kg of a compound of Formula I:

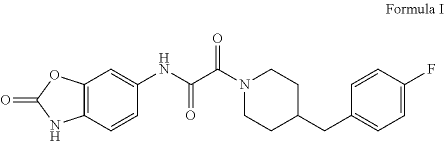 [SMILES: O=C(Nc1ccc2[nH]c(=O)oc2c1)C(=O)N1CCC(Cc2ccc(F)cc2)CC1]

(i) a solid pharmaceutically acceptable composition comprising: about 1% by weight of an anhydrous crystalline form of the compound of Formula I based on the total weight of the composition, wherein the anhydrous crystalline form has an X-ray powder diffraction pattern with characteristic peaks between and including the following values of 2θ in degrees: 6.4, 13.7, and 25.8±0.2° 2θ; about 70% to 89% by weight of a filler selected from the group consisting of mannitol, microcrystalline cellulose, and a combination thereof, based on the total weight of the composition; about 5% by weight of crospovidone based on the total weight of the composition; about 4% of povidone based on the total weight of the composition; and about 1% of a polysorbate based on the total weight of the composition; and (ii) an aqueous medium. In some embodiments, the aqueous medium comprises a starch-based suspension (e.g., SYRSPEND® SF).

### Methods of Use

The present disclosure provides, in an embodiment, methods of treating disorders in a subject comprising administering radiprodil, or pharmaceutically acceptable salt thereof, in a subject. In some embodiments, the subject is a pediatric subject.

In some embodiments, the disorder is an epileptic disorder. In some embodiments, the disorder is infantile spasm syndrome. In some embodiments, the disorder is a brain disorder characterized by a trait or state overactive glutamatergic transmission that include genetic disorders characterized by mutations in the NMDA glutamate receptor subunits as GRIN2B, GRIN2A, GRIN1 and GRIN2D, or other epileptic disorders determined by malformation of cortical development (e.g. Focal Cortical Dysplasia and Tuberous Sclerosis Complex) characterized by overexpression of the NDMA receptor subunit NR2B.

In one embodiment, provided herein is a method of treating a convulsive disorder in a subject in need thereof, comprising administering to the subject a pharmaceutical composition described herein or a pharmaceutically acceptable suspension described herein. In some embodiments, the convulsive disorder is epilepsy. In some embodiments, the subject is a pediatric subject. In some embodiments, the convulsive disorder is infantile spasm syndrome.

### Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

“Radiprodil” refers to the compound of Formula I as described herein and has the structure:

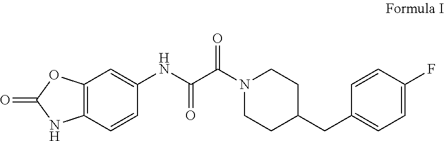 [SMILES: O=C(Nc1ccc2[nH]c(=O)oc2c1)C(=O)N1CCC(Cc2ccc(F)cc2)CC1]

Radiprodil is a negative allosteric modulator of the NMDA (N-methyl D-aspartate) receptor. The “radiprodil drug substance” described herein refers to a dihydrate of radiprodil.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: ACN: acetonitrile; LOQ: limit of quantification; NMT: not more than;

### Example 1. Synthesis of Radiprodil

An exemplary synthesis of radiprodil is provided in WO2003/010159, which is incorporated herein by reference.

### Example 2. Preparation of Form A of Radiprodil

An exemplary preparation and characterization of radiprodil Form A is provided in US Publication No. 2012/0059034, which is incorporated herein by reference.

### Example 3. Radiprodil Drug Gruanules

The formulation for pediatric use was developed as multiple unit oral dosage form. The approach was to develop a highly dispersible granule by wet granulation process in a fluid bed granulator to be reconstituted prior to administration.

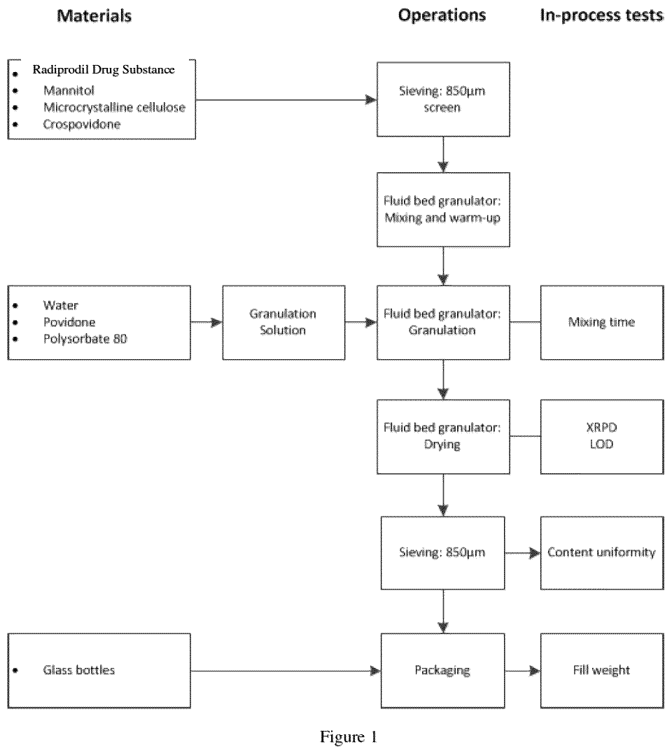

The manufacturing process for Radiprodil granules for oral suspension consists of a granulation in a fluid bed granulator followed by a filling of the final granules in bottle. The Radiprodil granules are manufactured in accordance with current Good Manufacturing Practice (cGMP). The equipment described may be replaced by any equipment of similar performance. A flow diagram of the manufacturing process for Radiprodil granules for oral suspension is presented as FIG. 1.

Different granule prototypes were manufactured using different qualitative compositions in terms of filler, surfactant, binder and disintegrant, always using suitable excipients for pediatric formulation. During the formulation development, it has been observed that all the granule prototypes produced via fluid bed granulation were containing Form A of radiprodil. Therefore the manufacturing process was optimized to allow complete conversion of the dihydrate form to Form A through the drying step at the end of granulation process.

The manufacturing formula given below corresponds to the theoretical batch size of 1 kg of granules. For other batch sizes the quantities of all ingredients will be adjusted proportionately.

The 1 kg batch formulas for 1% radiprodil and 10% radiprodil drug granules are provided in Tables 1 and 3 below, respectively. Bottle filling steps for 1% radiprodil and 10% radiprodil drug granules are provided in Tables 2 and 4, respectively.

TABLE 1
Manufacturing Formula—1% drug loading

| Raw Material | Amount (g) |
|---|---|
| Radiprodil Drug Substance | 10.9 |
| Mannitol (Pearlitol 100 SD) | 709.1 |
| Microcrystalline cellulose | 180.0 |
| (Avicel PH 101) | |
| Crospovidone (Kollidon CL) | 50.0 |
| Povidone (Kollidon 30) | 40.0 |
| Polysorbate 80 (Tween 80) | 10.0 |
| Purified water^{a} | 300.0 |
| Total (granules) | 1000.0 |

^{a}Removed through the process

TABLE 2
Manufacturing Formula for 1% drug
loading—Bottle filling step

| Raw Material | Amount |
|---|---|
| Granules 1% drug loading | 1000 mg^{b} |
| Bottle, 60 mL, round, glass, Type III, amber | 1 |
| CAP, Child Proof, polypropylene, white | 1 |

^{b}The weight is adapted based on the assay of the granules

TABLE 3
Manufacturing Formula—10% drug loading

| | Amount |
|---|---|
| Raw Material | (g) |
| Radiprodil Drug Substance | 109.0 |
| Mannitol (Pearlitol 100 SD) | 631.0 |
| Microcrystalline cellulose | 160.0 |
| (Avicel PH 101) | |
| Crospovidone (Kollidon CL) | 50.0 |
| Povidone (Kollidon 30) | 40.0 |
| Polysorbate 80 (Sorbitol 80 T 80 PH) | 10.0 |
| Purified water^{a} | 300.0 |
| Total (granules) | 1000.0 |

^{a}Removed through the process

TABLE 4
Manufacturing Formula—Bottle filling step

| Raw Material | Amount |
|---|---|
| Granules 10% drug loading | 1000 mg^{b} |
| Bottle, 60 mL, round, glass, | 1 |
| Type III, amber | |
| CAP, Child Proof, polypropylene, white | 1 |

^{b}The weight is adapted based on the assay of the granules

Scale-up of the granulation process in a fluid bed granulator was carried out and granules were manufactured at 1 kg scale. Process conditions were set with the aim to obtain full conversion of the dihydrate form (radiprodil drug substance) to Form A with a check of the XRPD pattern of the final granules. The trials demonstrated the feasibility of such a procedure to obtain a final product containing only the Form A.

Finally, one GMP batch at 10% drug loading was successfully manufactured at 1 kg scale for an ICH stability study by applying process conditions from scale-up trials.

### Example 3. 1% Radiprodil Granules

From the processes described in Example 3, radiprodil drug product is supplied as granules in bottles for oral suspension. Granules at 1% radiprodil drug loading are filled in 60 mL round amber Type III glass bottles with polypropylene childproof caps.

The quantitative composition of components used in radiprodil 1% drug product granules is provided in Table 5. All excipients utilized are standard compendial excipients commonly used in granulation processes.

TABLE 5

| | | | |
|---|---|---|---|
| | Quality | | Quantity |
| Component | Standard | Function | (mg) |

Granules

| | | | |
|---|---|---|---|
| Radiprodil Drug | In-house | Drug Substance | 10.9 |
| Substance | specification | | |
| Mannitol (Pearlitol | Ph. Eur. | Co-filler | 709.1 |
| 100SD) | | | |
| Microcrystalline | Ph. Eur, | Filler | 180.0 |
| cellulose (Avicel | | | |
| PH101) | | | |
| Crospovidone | Ph. Eur. | Disintegrant | 50.0 |
| (KollidonCL) | | | |
| Povidone (Kollidon | Ph. Eur. | Binder | 40.0 |
| 30) | | | |
| Polysorbate 80 | Ph. Eur. | Surfactant | 10.0 |
| (Tween 80) | | | |
| Total (granules) | | | 1000.0 |

Product

1000 mg
granules in
a 60 mL amber
glass bottle

### Example 4. 10% Radiprodil Granules

From the processes described in Example 3, radiprodil drug product is supplied as granules in bottles for oral suspension. Granules at 10% drug loading are filled in 60 mL round amber Type III glass bottles with polypropylene child-proof caps.

The quantitative composition of components used in radiprodil 10% drug product is provided in Table 6. All excipients utilized are standard compendial excipients commonly used in granulation processes.

TABLE 6

| | | | |
|---|---|---|---|
| | Quality | | Quantity |
| Component | Standard | Function | (mg) |

Granules

| | | | |
|---|---|---|---|
| Radiprodil Drug | In-house | Drug Substance | 109.0 |
| Substance | specification | | |
| Mannitol (Pearlitol | Ph, Eur. | Co-filler | 631.0 |
| 100SD) | | | |
| Microcrystalline | Ph. Eur. | Filler | 160.0 |
| cellulose (Avicel | | | |
| PH 101) | | | |
| Crospovidone | Ph. Eur. | Disintegrant | 50.0 |
| (Kollidon CL) | | | |
| Povidone (Kollidon | Ph. Eur. | Binder | 40.0 |
| 30) | | | |
| Polysorbate 80 | Ph. Eur. | Surfactant | 10.0 |
| (Sorbitol 80 T 80 PH) | | | |
| Total (granules) | | | 1000.0 |

Product

1000 mg
granules in
a 60 mL
amber glass
bottle

### Example 5. Suspensions of 1% and 10% Radiprodil Granules

1% and 10% radiprodil drug loading granules (Examples 2 and 3, respectively) are be reconstituted as a suspension prepared extemporaneously by adding 40 mL of diluent (4 mL of drinking water and 36 mL of SYRSPEND® SF).

### Example 6. Impurity and Stability Analysis of Radiprodil Drug Granules

Impurities in the radiprodil drug substance and granules were analyzed.

HPLC-DAD method is used for the identification of radiprodil. The retention time and the DAD spectrum must have same features as the ones obtained with a reference standard of radiprodil.

An HPLC method is used for the assay of radiprodil and for the determination of the degradation products in Radiprodil granules for oral suspension. The quantification of the drug substance is performed by comparing the peak areas of the sample with the corresponding peaks of the reference solution (external standard method). The quantification of the degradation products is performed in area percentage taking the sum of all the peaks that are ≥0.05% and that are not contained in the chromatogram of the blank solution or coming from the excipients.

HPLC method is used to determine the level of the impurity 6-amino-2-benzoxazolone in radiprodil granules for oral suspension. The quantification is performed by comparing the peak area of the sample with the peak of the reference solution (external standard method).

The potential and observed organic impurities in radiprodil drug substance batches are listed in Table 7.

TABLE 7
Impurities relating to radiprodil drug substance.

| Impurity | Structure/Formula/Molecular Weight | Source |
|---|---|---|
| Impurity 1 | 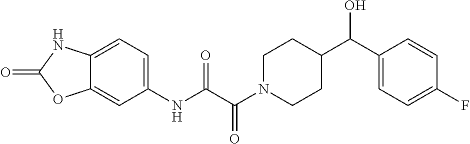 [SMILES: O=C(Nc1ccc2[nH]c(=O)oc2c1)C(=O)N1CCC(C(O)c2ccc(F)cc2)CC1] | Synthesis |
| | C₂₁H₂₀FN₃O₅ | |
| | 413.41 | |
| Impurity 2 | 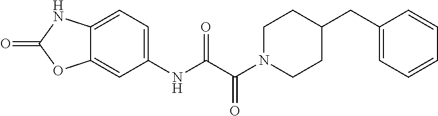 [SMILES: O=C(Nc1ccc2[nH]c(=O)oc2c1)C(=O)N1CCC(Cc2ccccc2)CC1] | Synthesis |
| | C₂₁H₂₁N₃O₄ | |
| | 379.42 | |
| Impurity 3 ([4-(4-Fluorobenzyl)- piperidin-1-yl]-oxo- acetic acid) | 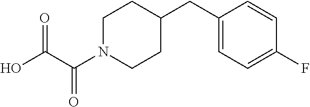 [SMILES: O=C(O)C(=O)N1CCC(Cc2ccc(F)cc2)CC1] | Starting Material |
| | C₁₄H₁₆FNO₃ | |
| | 265.29 | |
| Impurity 4 (6-Amino-2- benzoxazolone) | 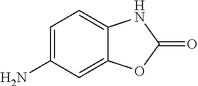 [SMILES: Nc1ccc2[nH]c(=O)oc2c1] | Starting Material |
| | C₇H₆N₂O₂ | |
| | 150.14 | |
| Impurity 5 (6-Nitro-2- benzoxazolone) | 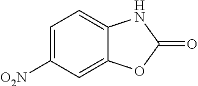 [SMILES: O=c1[nH]c2ccc([N+](=O)[O-])cc2o1] | Precursor of Starting Material |
| | C₇H₄N₂O₄ | |
| | 180.12 | |

Stability and impurity analyses of 1% and 10% granules, such as those described in Examples 3 and 4, respectively, and reconstituted formulations were studied. Table 8 displays stability data and impurity profiles for 1% radiprodil granules stored at 25° C./60% relative humidity (RH) at various time points. Table 9 displays stability data and impurity profiles for 1% radiprodil granules stored at 40° C./75% RH at various time points.

TABLE 8
Data at 25° C./60% RH (10% radiprodil granule)

| | Acceptance | Time points (months) | | |
|---|---|---|---|---|
| Test | Criteria | 0 | 3 | 6 |
| Appearance | White to off-white | White | White | White |
| | granules | granules | granules | granules |
| Assay: Radiprodil | 90.0-110.0% | 94.9% | 93.6% | 93.0 |
| | of label claim | | | |
| Water content | Reported value | 1.3% | 1.4% | 1.8% |
| Solid state | XRPD pattern | Form A | Form A | Form A |
| characterization | to be reported | | | |
| of granules | | | | |
| Solid state | XRPD pattern | Complies | Complies | Complies |
| characterization | consistent with the | | | |
| after | reference XRPD | | | |
| resuspension | pattern | | | |
| Suspension | Complies with Ph. | Complies | Complies | Complies |
| reconstitution test | Eur. 2.9.40 | | | |
| | criteria | | | |
| Degradation | | | | |
| products^{a} | | | | |
| 6-Amino-2- | NMT 0.5% | <LOQ | <LOQ | <LOQ |
| benzoxazolone | | (0.05%) | (0.05%) | (0.05%) |
| Impurity 2 | NMT 0.5% | 0.06% | 0.06% | 0.06% |
| Impurity 1 | NMT 0.5% | <LOQ | <LOQ | <LOQ |
| | | (0.05%) | (0.05%) | (0.05%) |
| Impurity 3 | NMT 0.5% | <LOQ | <LOQ | <LOQ |
| | | (0.05%) | (0.05%) | (0.05%) |
| Any unspecified | NMT 0.2% | 0.07% | 0.06% | 0.05% |
| degradation | | | | |
| product | | | | |
| Total unspecified | NMT 1.0% | 0.06% | 0.07% | 0.05% |
| degradation | | | | |
| products | | | | |
| Total degradation | NMT 2.5% | 0.13% | 0.12% | 0.11% |
| products greater | | | | |
| than 0.05% | | | | |
| (6-amino-2- | | | | |
| benzoxazolone not | | | | |
| included) | | | | |

^{a}Degradation products are expressed in % claim

TABLE 9
Data at 40° C./75% RH (10% radiprodil granule)

| | Acceptance | Time points (months) | | | |
|---|---|---|---|---|---|
| Test | Criteria | 0 | 1 | 3 | 6 |
| Appearance | White to off- | White | White | White | White |
| | white granules | granules | granules | granules | granules |
| Assay: radiprodil | 90.0-110.0% of | 94.9% | 92.9% | 92.1% | 91.9% |
| | label claim | | | | |
| Water content | Reported value | 1.3% | 1.8% | 2.0% | 2.2% |
| Solid state | XRPD pattern to | Form A | Form A | Form A | Form A |
| characterization | be reported | | | | |
| of granules | | | | | |
| Solid state | XRPD pattern | Complies | Complies | Complies | Complies |
| characterization | consistent with the | | | | |
| after resuspension | reference XRPD | | | | |
| | pattern | | | | |
| Suspension | Complies with | Complies | Complies | Complies | Complies |
| reconstitution test | Ph. Eur. 2.9.40 | | | | |
| | criteria | | | | |
| Degradation products^{a} | | | | | |
| 6-Amino-2- | NMT 0.5% | <LOQ | <LOQ | <LOQ | <LOQ |
| benzoxazolone | | (0.05%) | (0.05%) | (0.05%) | (0.05%) |
| Impurity 2 | NMT 0.5% | 0.06% | 0.06% | 0.08% | 0.06% |
| Impurity 1 | NMT 0.5% | <LOQ | <LOQ | <LOQ | 0.05% |
| | | (0.05%) | (0.05%) | (0.05%) | |
| Impurity 3 | NMT 0.5% | <LOQ | <LOQ | <LOQ | <LOQ |
| | | (0.05%) | (0.05%) | (0.05%) | (0.05%) |
| Any unspecified | NMT 0.2% | 0.07% | 0.07% | 0.06% | 0.06% |
| degradation | | | | | |
| product | | | | | |
| Total unspecified | NMT 1.0% | 0.07% | 0.07% | 0.06% | 0.06% |
| degradation | | | | | |
| products | | | | | |
| Total degradation | NMT 2.5% | 0.13% | 0.13% | 0.14% | 0.17% |
| products greater | | | | | |
| than 0.05% | | | | | |
| (6-amino-2- | | | | | |
| benzoxazolone not | | | | | |
| included) | | | | | |

^{a}Degradation products are expressed in % claim

Table 10 displays stability data and impurity profiles for 10% radiprodil granules stored at 40° C./75% RH at various time points.

TABLE 10
Data at 40° C./75% RH (10% radiprodil granule)

| | | Time points (months) | |
|---|---|---|---|
| Test | Acceptance Criteria | 0 | 1 |
| Appearance | White to off-white | White | White |
| | granules | granules | powder |
| Assay: radiprodil content | 90.0-110.0% of label | 92.9% | 91.0% |
| | claim | | |
| Water content | Reported value | 1.2% | 1.7% |
| Solid state | XRPD pattern to be | Form A | Form |
| characterization of | reported | | A |
| granules | | | |
| Solid state | XRPD pattern | Complies | Complies |
| characterization | consistent | | |
| afterresuspension | with thereference | | |
| | XRPD pattern | | |
| Suspension | Complies with | Complies | Complies |
| reconstitution | Ph. Eur. | | |
| test | 2.9.40criteria | | |
| Degradation products^{a} | | | |
| 6-Amino-2- | NMT 0.5% | <LOQ | <LOQ |
| benzoxazolone | | (0.05%) | (0.05%) |
| Impurity 2 | NMT 0.5% | 0.05% | 0.05% |
| Impurity 1 | NMT 0.5% | <LOQ | <LOQ |
| | | (0.05%) | (0.05%) |
| Piperidinyl-oxo- | NMT 0.5% | <LOQ | <LOQ |
| acetic acid | | (0.05%) | (0.05%) |
| Any unspecified | NMT 0.2% | 0.06% | 0.07% |
| degradation | | | |
| product | | | |
| Total unspecified | NMT 1.0% | 0.06% | 0.07% |
| degradation | | | |
| products | | | |
| Total degradation | NMT 2.5% | 0.11% | 0.12% |
| products greater than | | | |
| 0.05% (6-amino-2- | | | |
| benzoxazolone | | | |
| not included) | | | |

^{a}Degradation products are expressed in % claim

### Example 7. Dissolution Profiles of Radiprodil Compositions

This study summarizes dissolution experiments preformed on the radiprodil drug substance and on the drug product including two drug loadings with and without reconstitution in SYRSPEND® and water prior to the dissolution test.

All dissolution experiments were performed on six vessels and the comparative curves were plotted using the average of them.

Some dissolution experiments were performed using prior to the dissolution a reconstitution in Syrpend® and water. In that case, the reconstitution was prepared and let under stirring for 1 night prior to the dissolution test. In order to mimic the clinical pharmacy manual, an additional test was performed with an extemporaneous suspension (15 min under stirring prior to the dissolution).

Dissolution and high performance liquid chromatography (HPLC) parameters used in the experiments are provided below in Tables 11 and 12, respectively.

TABLE 11
Dissolution Parameters

| Description | Value |
|---|---|
| Apparatus | USPII |
| | (paddle apparatus with Copley) |
| Dissolution medium | 0.5% sodium lauryl sulphate |
| | solution in water |
| Dissolution medium | 900 |
| volume (mL) | |
| Dissolution medium | 37 +/− 0.5° C. |
| temperature (° C.) | |
| Rotation speed (rpm) | 75 |
| Sampling time (min) | 5, 10, 15, 30, 45 and 60 |
| Sampling volume (mL) | NA (automated sampling) |
| Separative technique | Full flow filters (45 um) |

TABLE 12
HPLC Parameters

| Description | Value |
|---|---|
| Column type | Symmetry Shield RP-18 |
| | (15 mm * 4.6 mm − 3.5 μm) |
| Mobile phase | Mix of phase A/phase B = |
| | 45/55% v/v |
| Mobile phase (phase A) | Water/Acetonitrile (ACN)/ |
| | 1M TEAP (900:90:10 v/v/v) |
| Mobile phase (phase B) | Water/ACN/1M TEAP |
| | (90:900:10 v/v/v) |
| Flow rate (mL/min) | 1.0 |
| Column temperature (° C.) | 30 |
| Detector type | UV |
| Wavelength (nm) | 255 |
| Injection volume (μL) | 20 |

Dissolution Experiments without Reconstitution in SYRSPEND® SF and Water Prior to the Dissolution Tests.

Radiprodil, as the dihydrate and Form A, as well as Form A containing granules (1% and 10% as described above) and blends of dihydrate and Form A (1% and 10% blends of dihydrate form and Form A) were submitted to dissolution tests in sodium lauryl sulfate (0.5% in water).

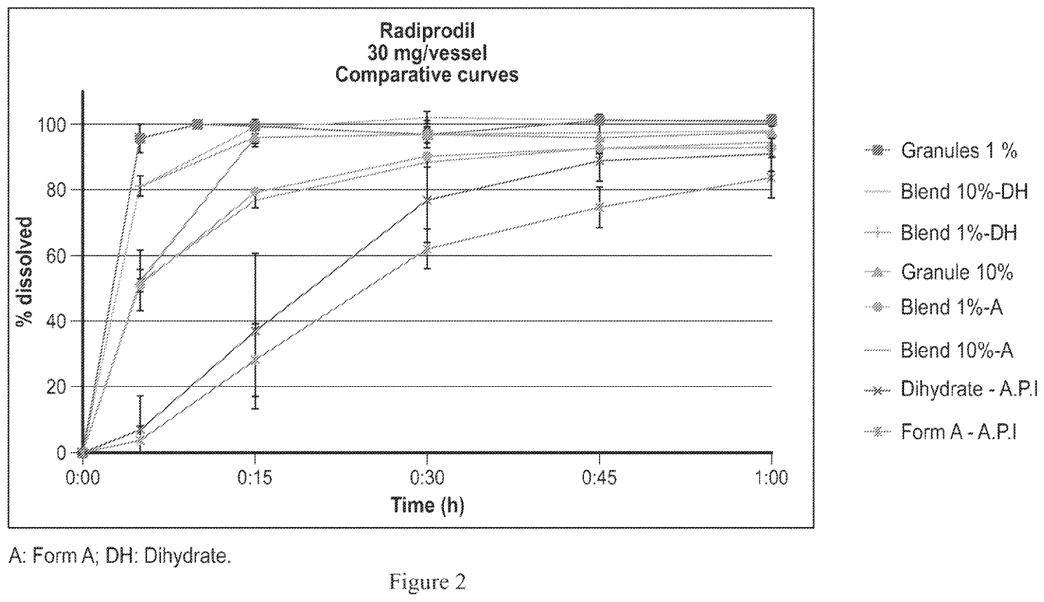

Exemplary dissolution profiles are provided in FIG. 2. The dissolution profile of the dihydrate form appears more rapid in aqueous media than the one of anhydrous form A. This observation supports the results of the stability study which shows that anhydrous form A is more stable than the dihydrate even in presence of water.

Dissolution Experiments with Reconstitution in SYRSPEND® SF and Water Prior to Dissolution Tests

Radiprodil, as the dihydrate and Form A, as well as Form A containing granules (1% and 10% as described above) and blends of dihydrate and Form A (1% and 10% blends of dihydrate form and Form A) were first reconstituted in a mix of SYRSPEND® and water and stirred for 24 hours. The suspensions were subsequently submitted to dissolution tests.

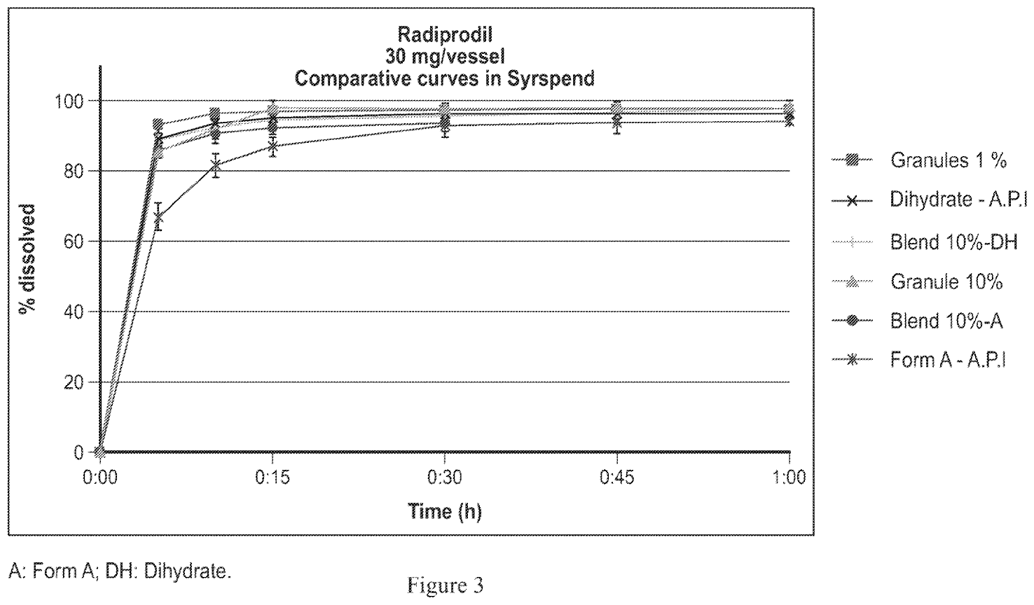

Exemplary dissolution profiles are provided in FIG. 3. For blends reconstituted in mixture SYRSPEND® and water prior to the dissolution test (1 night under stirring), no difference is observed between the 10% blends having the dihydrate form or anhydrous Form A. This observation reveals that the reconstitution in SYRSPEND® and water allows smoothing out the difference from the solid form of the radiprodil drug substance (probably due to a better wettability and homogeneity).

Dissolution Experiments with Extemporaneous Reconstitutionin SYRSPEND® SF and Water Prior to the Dissolution Tests

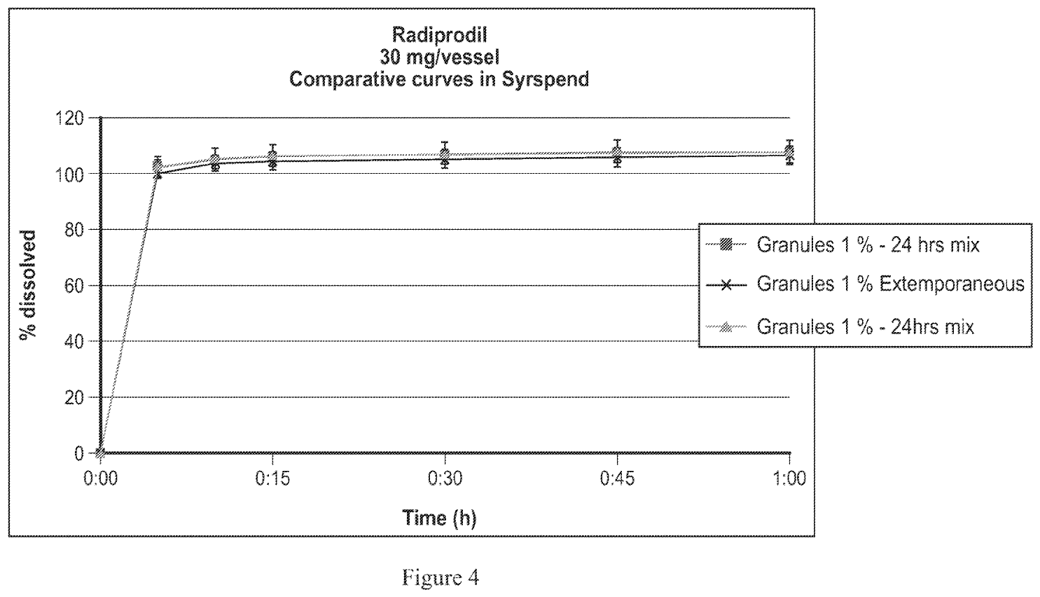

To evaluate the impact of the time of a reconstituted solution, an extemporaneous solution of 1% Radiprodil Form A granules in SYRSPEND® and water has been prepared 15 min before the dissolution test and was compared to 1% Radiprodil Form A granules reconstituted in SYRSPEND® and water for 24 hours. Exemplary profiles are shown in FIG. 4. No differences on the dissolution profiles were observed between granules extemporaneously reconstituted or reconstituted for 24 hours prior to the dissolution test.

### Example 8. Preparation of Form C of Radiprodil

An exemplary preparation and characterization of radiprodil Form C is provided in US Publication No. 2012/0010044, which is incorporated herein by reference.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1-45. (canceled)

46. A method of treating an epileptic disorder in a human patient in need thereof, comprising administering to the patient a pharmaceutically acceptable suspension comprising: (i) a therapeutically effective amount of a compound of Formula I: 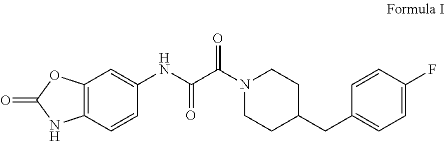 [SMILES: O=C(Nc1ccc2[nH]c(=O)oc2c1)C(=O)N1CCC(Cc2ccc(F)cc2)CC1]
or a pharmaceutically acceptable salt thereof, (ii) a pharmaceutically acceptable excipient, and (iii) 6-amino-2-benzoxazolone, wherein the 6-amino-2-benzoxazolone is present in an amount of not more than 0.05% with respect to the quantity of the compound of Formula I.

47. The method of claim 46, wherein the 6-amino-2-benzoxazolone is present in an amount of not more than 0.05% with respect to the quantity of the compound of Formula I when exposed to 60% relative humidity at 25° C. for about 6 months.

48. The method of claim 46, wherein the compound is characterized by an X-ray powder diffraction pattern with characteristic peaks between and including the following values of 2θ in degrees: 7.8, 22.0, 23.7, 27.0 and 27.6±0.2° 2θ (Form A).

49. The method of claim 46, wherein the compound is characterized by an X-ray powder diffraction pattern with characteristic peaks between and including the following values of 2θ in degrees: 6.4, 13.7, and 25.8±0.2° 2θ (Form C).

50. The method of claim 48, wherein the pharmaceutically acceptable suspension further comprises an aqueous medium.

51. The method of claim 50, wherein the aqueous medium comprises a starch-based suspension.

52. The method of claim 46, wherein the epileptic disorder is determined by a malformation of cortical development.

53. The method of claim 52, wherein the malformation of cortical development is focal cortical dysplasia or tuberous sclerosis complex.

54. A method of treating a brain disorder characterized by overactive glutamatergic transmission in a human patient in need thereof, comprising administering to the patient a pharmaceutically acceptable suspension comprising: (i) a therapeutically effective amount of a compound of Formula I: 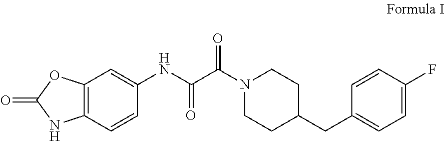 [SMILES: O=C(Nc1ccc2[nH]c(=O)oc2c1)C(=O)N1CCC(Cc2ccc(F)cc2)CC1]
or a pharmaceutically acceptable salt thereof, (ii) a pharmaceutically acceptable excipient, and (iii) 6-amino-2-benzoxazolone, wherein the 6-amino-2-benzoxazolone is present in an amount of not more than 0.05% with respect to the quantity of the compound of Formula I.

55. The method of claim 54, wherein the brain disorder is characterized by a mutation in an NMDA glutamate receptor subunit selected from the group consisting of GRIN2B, GRIN2A, GRIN1 and GRIN2D.

56. The method of claim 54, wherein the 6-amino-2-benzoxazolone is present in an amount of not more than 0.05% with respect to the quantity of the compound of Formula I when exposed to 60% relative humidity at 25° C. for about 6 months.

57. The method of claim 54, wherein the compound is characterized by an X-ray powder diffraction pattern with characteristic peaks between and including the following values of 2θ in degrees: 7.8, 22.0, 23.7, 27.0 and 27.6±0.2° 2θ (Form A).

58. The method of claim 54, wherein the compound is characterized by an X-ray powder diffraction pattern with characteristic peaks between and including the following values of 2θ in degrees: 6.4, 13.7, and 25.8±0.2° 2θ (Form C).

59. The method of claim 57, wherein the pharmaceutically acceptable suspension further comprises an aqueous medium.

60. The method of claim 59, wherein the aqueous medium comprises a starch-based suspension.

61. A method of treating a brain disorder characterized by a mutation in GRIN2B, GRIN2A, GRIN1, or GRIN2D in a human patient in need thereof, comprising administering to the patient a pharmaceutically acceptable suspension comprising: a therapeutically effective amount of a compound of Formula I: 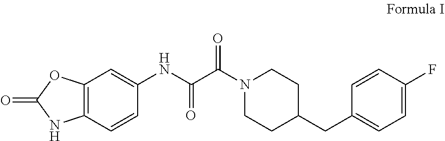 [SMILES: O=C(Nc1ccc2[nH]c(=O)oc2c1)C(=O)N1CCC(Cc2ccc(F)cc2)CC1]
or a pharmaceutically acceptable salt thereof, and a pharmaceutically acceptable excipient, wherein 6-amino-2-benzoxazolone is present in the pharmaceutically acceptable suspension in an amount of not more than 0.05% with respect to the quantity of the compound of Formula I.

62. The method of claim 61, wherein the 6-amino-2-benzoxazolone is present in an amount of not more than 0.05% with respect to the quantity of the compound of Formula I when exposed to 60% relative humidity at 25° C. for about 6 months.

63. The method of claim 61, wherein the compound is characterized by an X-ray powder diffraction pattern with characteristic peaks between and including the following values of 2θ in degrees: 7.8, 22.0, 23.7, 27.0 and 27.6±0.2° 2θ (Form A).

64. The method of claim 61, wherein the compound is characterized by an X-ray powder diffraction pattern with characteristic peaks between and including the following values of 2θ in degrees: 6.4, 13.7, and 25.8±0.2° 2θ (Form C).

65. The method of claim 63, wherein the pharmaceutically acceptable suspension further comprises an aqueous medium.

66. The method of claim 65, wherein the aqueous medium comprises a starch-based suspension.

67. A method of treating a brain disorder characterized by a mutation in GRIN2B, GRIN2A, GRIN1, or GRIN2D in a human patient in need thereof, comprising administering to the patient a pharmaceutically acceptable suspension comprising: (i) a therapeutically effective amount of a compound of Formula I: 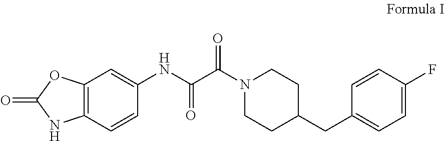 [SMILES: O=C(Nc1ccc2[nH]c(=O)oc2c1)C(=O)N1CCC(Cc2ccc(F)cc2)CC1]
or a pharmaceutically acceptable salt thereof, (ii) a pharmaceutically acceptable excipient, and (iii) 6-amino-2-benzoxazolone, wherein the 6-amino-2-benzoxazolone is present in an amount of not more than 0.05% with respect to the quantity of the compound of Formula I.

68. The method of claim 67, wherein the 6-amino-2-benzoxazolone is present in an amount of not more than 0.05% with respect to the quantity of the compound of Formula I when exposed to 60% relative humidity at 25° C. for about 6 months.

69. The method of claim 67, wherein the compound is characterized by an X-ray powder diffraction pattern with characteristic peaks between and including the following values of 2θ in degrees: 7.8, 22.0, 23.7, 27.0 and 27.6±0.2° 2θ (Form A).

70. The method of claim 67, wherein the compound is characterized by an X-ray powder diffraction pattern with characteristic peaks between and including the following values of 2θ in degrees: 6.4, 13.7, and 25.8±0.2° 2θ (Form C).

71. The method of claim 69, wherein the pharmaceutically acceptable suspension further comprises an aqueous medium.

72. The method of claim 71, wherein the aqueous medium comprises a starch-based suspension.
